(19) United States
(12) Patent Application Publication
(10) Pub. No.: US 2016/0050989 A1
(43) Pub. Date: Feb. 25, 2016

(54) SPORTS PROTECTIVE GEAR AND AUXILIARY DEVICE THEREFOR

(71) Applicant: Wellpower Sporting Goods Co., Ltd., Hong Kong (CN)

(72) Inventor: Shing-Jiu Sheu, Taipei City (TW)

(73) Assignee: Wellpower Sporting Goods Co., Ltd.

(21) Appl. No.: 14/467,850

(22) Filed: Aug. 25, 2014

(51) Int. Cl. A41D 13/06; A61F 13/06

(52) U.S. Cl. CPC A41D 13/065; A61F 13/062; A41D 2600/10; A63B 2071/125

(57) ABSTRACT

A sports protective gear includes a stretchable protective sleeve defining a wearing space for wearing by a user and having a through hole bounded by a boundary edge of the protective sleeve, and an auxiliary device including a finger operable member disposed in the through hole and having a looped wall and two spaced-apart looped flanges. The looped wall defines a finger pass age. The flanges project outwardly and respectively from inner and outer peripheries of the looped wall, and cooperatively define therebetween an engaging groove which extends around the looped wall and which engages the boundary edge of the protective sleeve. The flanges respectively abut against inner and outer surfaces of the protective sleeve.

### FIELD OF THE INVENTION

The invention relates to a sports protective gear, more particularly to a sports protective gear that can enhance support of a joint and an auxiliary device therefor.

### BACKGROUND OF THE INVENTION

Referring to FIG. 1, a conventional sports protective gear 1 for a knee includes a hollow protective main body 11 having a top opening 12 at a top end thereof and a bottom opening 13 at a bottom end thereof.

To wear the protective gear 1 on the user's knee, the user pinches a top end portion of the protective main body 11 and pulls it upward until the protective main body 11 is fitted over the knee of the user. However, after repeated wear and removal of the protective gear 1, the top opening 12 is easily stretched out, and becomes larger and larger, so that the protecting effect of the protective main body 11 is lost.

### SUMMARY OF THE INVENTION

Therefore, the object of the present invention is to provide a sports protective gear that is capable of overcoming the aforementioned drawbacks of the prior art.

Accordingly, a sports protective gear comprises a protective main body and an auxiliary device. The protective main body includes a stretchable protective sleeve that defines a wearing space for wearing by a user and that has a through hole which extends through inner and outer surfaces thereof to communicate with the wearing space and which is bounded by a boundary edge of the protective sleeve. The auxiliary device includes a finger operable member disposed in the through hole. The finger operable member includes a looped wall and two spaced-apart looped flanges. The looped wall defines a finger passage for a finger of a user to extend therethrough, and has an inner periphery proximal to the wearing space, and an outer periphery distal from the wearing space. The looped flanges project outwardly and respectively from the inner and outer peripheries of the looped wall, and cooperatively define therebetween an engaging groove which extends around the looped wall and which engages the boundary edge of the protective sleeve. The looped flanges respectively abut against the inner and outer surfaces of the protective sleeve.

Another object of this invention is to provide an auxiliary device for the aforesaid sports protective gear.

Accordingly, an auxiliary device is used for a sports protective gear having a protective main body that includes a stretchable protective sleeve. The protective sleeve defines a wearing space for wearing by a user and has a through hole which extends through inner and outer surfaces of the protective sleeve to communicate with the wearing space and which is bounded by a boundary edge of the protective sleeve. The auxiliary device comprises a finger operable member for disposal in the through hole and having a looped wall and two spaced-apart looped flanges. The looped wall defines a finger passage for a finger of a user to extend therethrough, and has an inner periphery adapted to be proximal to the wearing space, and an outer periphery that is adapted to be distal from the wearing space. The looped flanges project outwardly and respectively from the inner and outer peripheries of the looped wall, and cooperatively define therebetween an engaging groove which extends around the looped wall and which is adapted to engage the boundary edge of the protective sleeve. The looped flanges are adapted to respectively abut against the inner and outer surfaces of the protective sleeve.

### BRIEF DESCRIPTION OF THE DRAWINGS

Other features and advantages of the present invention will become apparent in the following detailed description of the embodiments with reference to the accompanying drawings, of which:

FIG. 1 illustrates a conventional protective gear in a state of use;

FIG. 2 is a perspective view of a sports protective gear according to the first embodiment of the present invention;

FIG. 3 is a perspective view of a finger operable member of the first embodiment;

FIG. 4 is a sectional view of the first embodiment taken along line IV-IV of FIG. 2;

FIG. 5 illustrates how a user wears the protective gear of the first embodiment;

FIG. 6 illustrates how a user wears a sports protective gear according to the second embodiment of this invention; and

FIG. 7 illustrates how a user wears a sports protective gear according to the third embodiment of this invention.

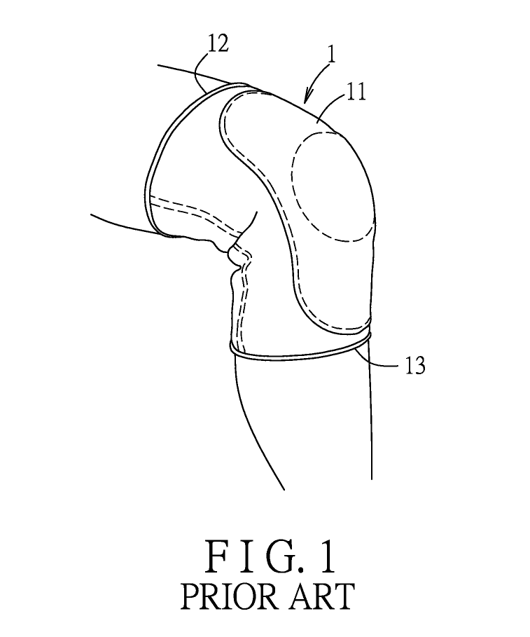

### DETAILED DESCRIPTION OF THE EMBODIMENTS

Before the present invention is described in greater detail, it should be noted that the same reference numerals have been used to denote like elements throughout the specification.

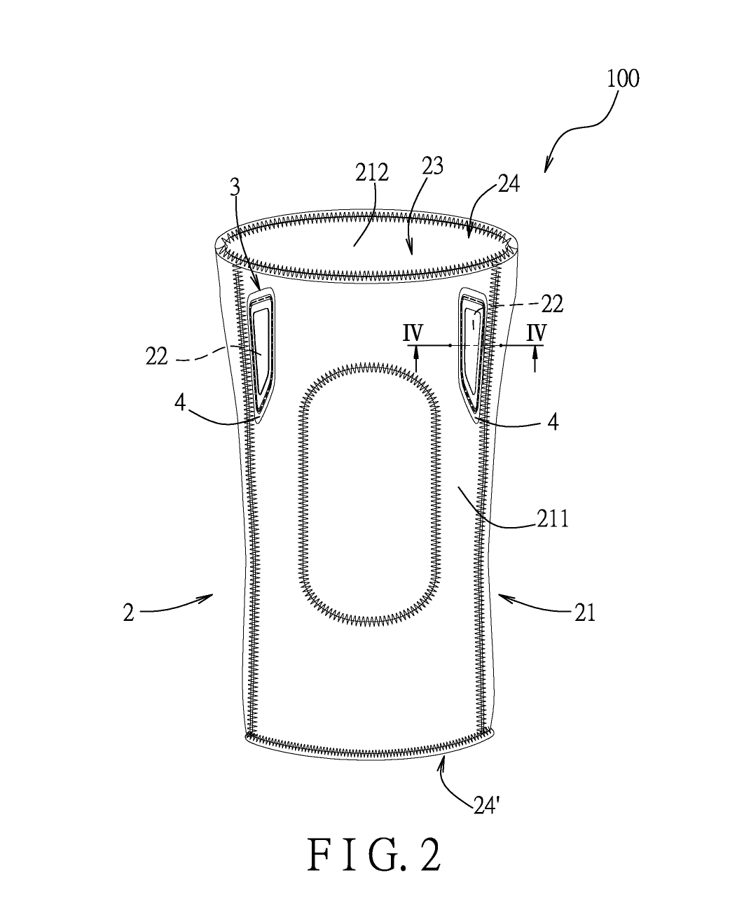

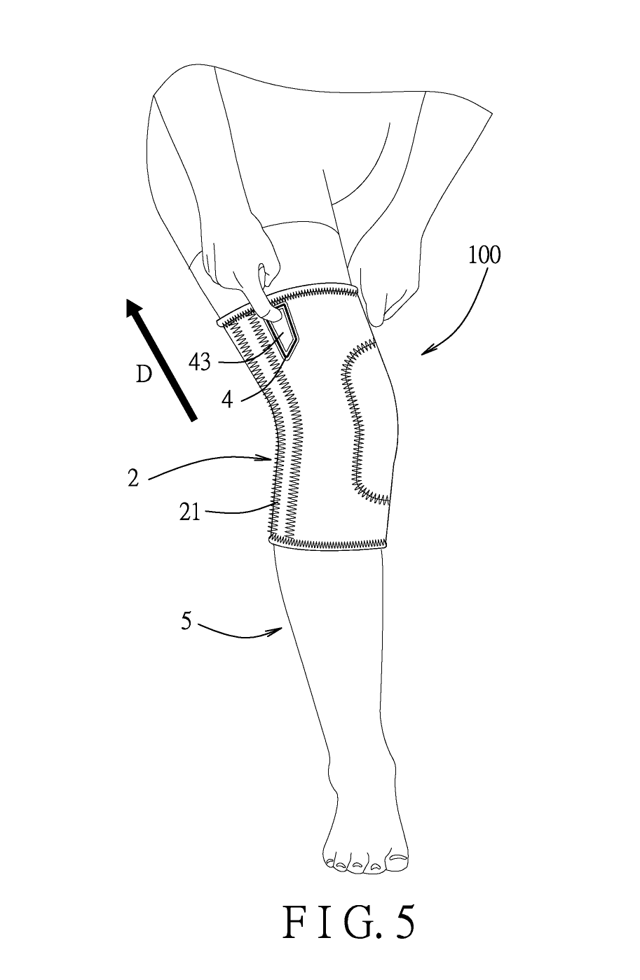

Referring to FIGS. 2 to 4, a sports protective gear 100 according to the first embodiment of the present invention is configured to be worn on a knee of a user 5 (see FIG. 5), and is shown to comprise a protective main body 2 and an auxiliary device 3.

The protective main body 2 includes a stretchable protective sleeve 21 having an outer surface 211 and an inner surface 212 opposite to the outer surface 211, and two through holes 22 extending through the inner and outer surfaces 212, 211 and spaced apart in a left-right direction. The inner surface 212 defines a wearing space 23 for wearing by the user 5. The wearing space 23 communicates with the through holes 22. In this embodiment, the protective sleeve 21 is tubular, and has a top opening 24 at a top end thereof, and a bottom opening 24′ at a bottom end thereof. The through holes 22 are proximate to the top opening 24. Each through hole 22 is bounded by a boundary edge 221 of the protective sleeve 21.

The auxiliary device 3 includes two finger operable members 4 respectively disposed in the through holes 22. The two through holes 22 have an identical shape, that is, a substantially trapezoidal shape. The finger operable members 4 have a similar structure and shape. The shape of each through hole 22 is not limited to the trapezoidal shape, but maybe oval, rectangular, or other shapes. The shape of each finger operable member 4 is adjusted to correspond to that of each through hole 22. To facilitate description of this embodiment, only one finger operable member 4 will be described hereinbelow.

With reference to FIGS. 2 to 4, the finger operable member 4 includes a looped wall 41 and two spaced-apart looped flanges 42. The looped wall 41 defines a finger passage 43 for a finger of the user 5 to extend therethrough, and has an inner periphery 410 proximal to the wearing space 23, and an outer periphery 410′ distal from the wearing space 23. The finger passage 43 communicates with the wearing space 23. The looped flanges 42 project outwardly and respectively from the inner and outer peripheries 410, 410′ of the looped wall 41, and cooperatively define therebetween an engaging groove 44 which extends around the looped wall 41 and which engages the boundary edge 221 of the protective sleeve 21. The looped flanges 42 respectively abut against the inner and outer surfaces 212, 211 of the protective sleeve 21.

Specifically, the looped wall 41 has an inner wall surface 411 defining the finger passage 43, and an outer wall surface 412 opposite to the inner wall surface 411. Each of the looped flanges 42 projects perpendicularly from the respective one of the inner and outer peripheries 410, 410′ away from the inner wall surface 411, and has a substantially trapezoidal shape. The looped flange 42 projecting from the outer periphery 410′ has a looped stitch groove 421 formed in an outer surface 420 thereof. A stitching line (S) is sewn in the looped stitch groove 421 to fix the finger operable member 4 to the protective sleeve 21.

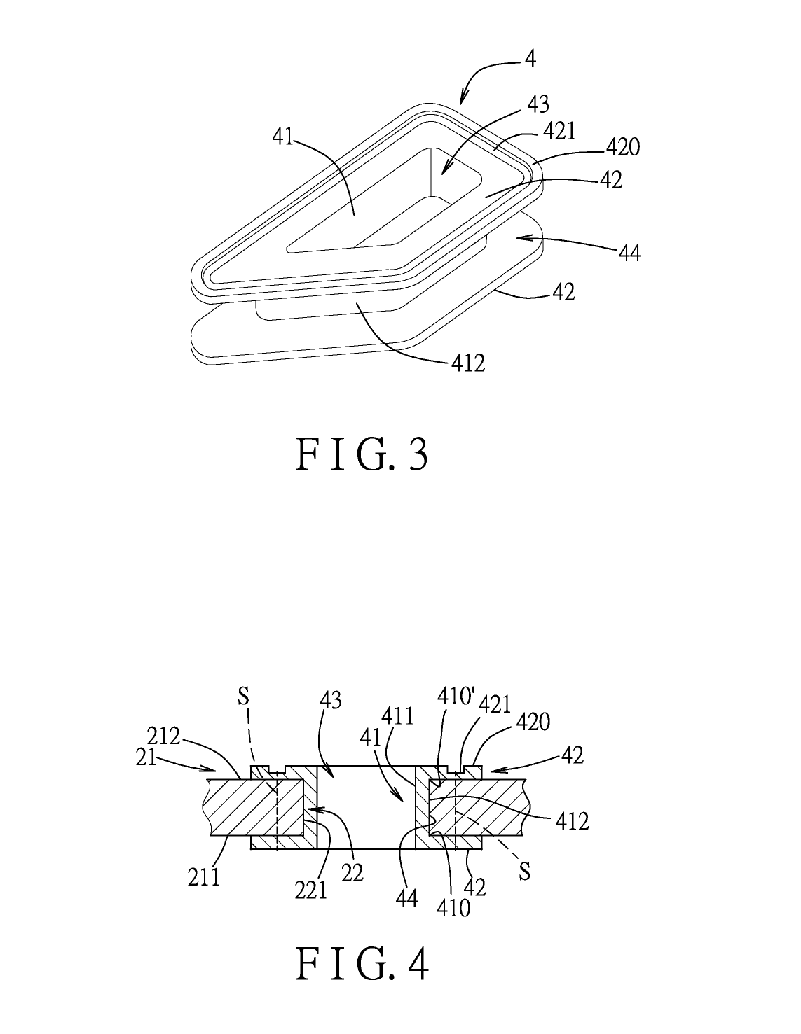

Referring to FIG. 5, in combination with FIGS. 2 and 3, when the user 5 desires to wear the protective gear 100, a finger of both hands of the user 5 is inserted into the finger passage 43 of the respective finger operable member 4, after which the fingers of both hands of the user 5 pull upward the looped walls 41 of the finger operable members 4 along a pull direction (D) so as to move the protective sleeve 21 of the protective gear 100 through the foot, the leg, the knee and the thigh of the user 5 until the protective sleeve 21 is tightly fitted around the knee of the user 5.

During wear of the protective gear 100, the finger passage 43 of each finger operable member 4 facilitates application of force by a finger of the user 5, so that the force is not directly applied to the protective sleeve 21 and the top opening 24 of the protective main body 2 is not easily stretched out. Hence, the tightening function of the protective sleeve 21 is maintained.

It is worth to mention that since one of the looped flanges 42 may be in contact with the skin of the user 5, the auxiliary device 3 is thus made of a material which will not produce a strange feeling with the skin of the user 5, such as a plastic or soft plastic material.

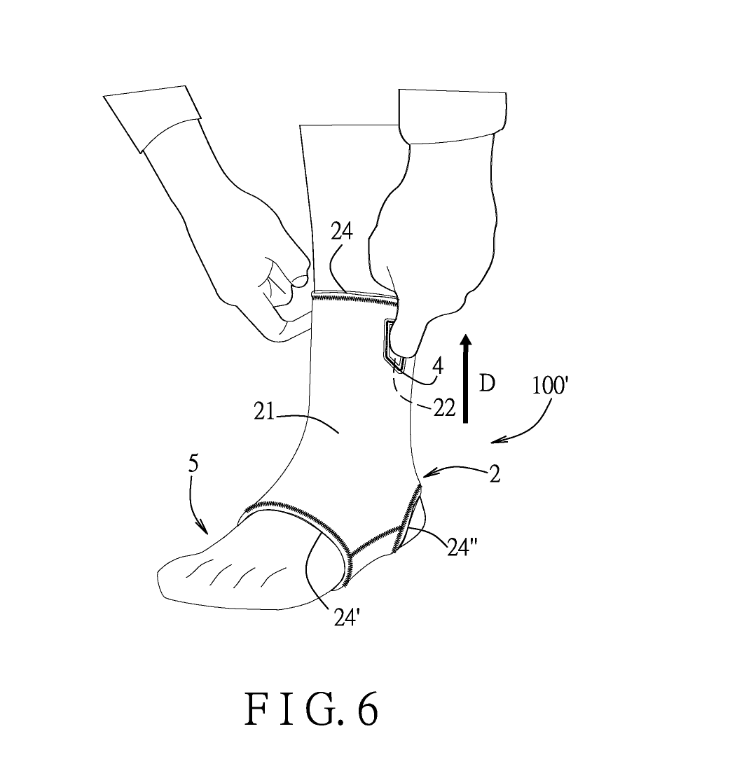

Referring to FIG. 6, a sports protective gear 100′ according to the second embodiment of the present invention is configured to be worn on an ankle of the user 5, and is shown to be substantially similar to that of the first embodiment. However, in this embodiment, the protective sleeve 21 is substantially L-shaped, and further has a heel opening 24″ between the top and bottom openings 24, 24′. The wearing method of the protective gear 100′ is similar to that described in the first embodiment.

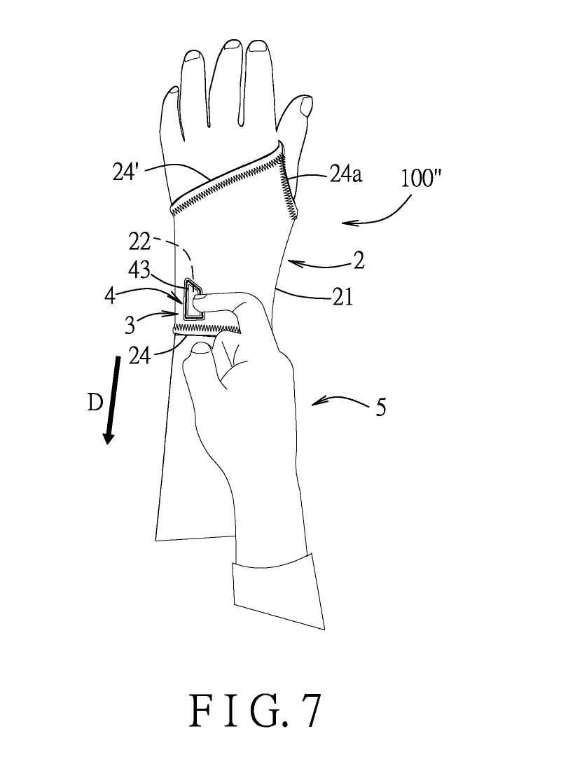

Referring to FIG. 7, a sports protective gear 100″ according to the third embodiment of the present invention is configured to be worn on a wrist of the user 5, and is shown to be substantially similar to that of the first embodiment. However, in this embodiment, the protective sleeve 21 further has a thumb opening (24a) adjacent to the bottom opening 24′, and has only one through hole 22 proximate to the top opening 24. Further, the auxiliary device 3 has only one finger operable member 4.

The wearing method of the protective gear 100″ is similar to that described in the first embodiment. The difference resides in that when a left hand of the user 5 wears the protective gear 100″, a finger of the right hand of the user 5 is used to pull the finger operable member 4 along the pull direction (D) to accomplish wearing of the protective gear 100″.

The configuration of the protective gear 100, 100′, 100″ is not limited to a knee protective gear 100 shown in FIG. 5, an ankle protective gear 100′ shown in FIG. 6, and a wrist protective gear 100″ shown in FIG. 7, but may also be an elbow protective gear (not shown). Moreover, the number of the finger operable member 4 may be increased or decreased depending on the configuration of the protective gear.

In summary, through the finger passage 43 of each finger operable member 4 that facilitates application of force by a finger of the user 5, the force is not directly applied to the protective sleeve 21 and the top opening 24 of the protective main body 2 is not easily stretched out, so that the tightening function of the protective sleeve 21 is maintained. Hence, the object of this invention can be achieved.

While the present invention has been described in connection with what are considered the most practical embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A sports protective gear comprising:
a protective main body including a stretchable protective sleeve that defines a wearing space for wearing by a user and that has a through hole which extends through inner and outer surfaces of said protective sleeve to communicate with said wearing space and which is bounded by a boundary edge of said protective sleeve; and
an auxiliary device including a finger operable member disposed in said through hole, said finger operable member including a looped wall and two spaced-apart looped flanges, said looped wall defining a finger passage for a finger of a user to extend therethrough, and having an inner periphery proximal to said wearing space, and an outer periphery distal from said wearing space, said looped flanges projecting outwardly and respectively from said inner and outer peripheries of said looped wall, and cooperatively defining therebetween an engaging groove which extends around said looped wall and which engages said boundary edge of said protective sleeve, said looped flanges respectively abutting against said inner and outer surfaces of said protective sleeve.

2. The sports protective gear of claim 1, wherein said finger passage communicates with said wearing space, said looped wall having an inner wall surface defining said finger passage, and an outer wall surface opposite to said inner wall surface, each of said looped flanges projecting from a respective one of said inner and outer peripheries of said looped wall away from said inner wall surface.

3. The sports protective gear of claim 1, wherein said protective sleeve is formed with a plurality of spaced-apart openings communicating with said wearing space, said through hole being proximate to one of said openings.

4. The sports protective gear of claim 2, wherein said looped flanges are respectively fixed to said inner and outer surfaces of said protective sleeve.

5. The sports protective gear of claim 2, wherein one of said looped flanges has a looped stitch groove formed in an outer surface thereof.

6. An auxiliary device for a sports protective gear having a protective main body that includes a stretchable protective sleeve, the protective sleeve defining a wearing space for wearing by a user and having a through hole which extends through inner and outer surfaces of the protective sleeve to communicate with the wearing space and which is bounded by a boundary edge of said protective sleeve, said auxiliary device comprising:
a finger operable member for disposal in said through hole and having a looped wall and two spaced-apart looped flanges, said looped wall defining a finger passage for a finger of a user to extend therethrough, and having an inner periphery adapted to be proximal to the wearing space, and an outer periphery that is adapted to be distal from the wearing space, said looped flanges projecting outwardly and respectively from said inner and outer peripheries of said looped wall, and cooperatively defining therebetween an engaging groove which extends around said looped wall and which is adapted to engage the boundary edge of the protective sleeve, said looped flanges being adapted to respectively abut against the inner and outer surfaces of the protective sleeve.

7. The auxiliary device of claim 6, wherein said finger passage is adapted to communicate with the wearing space, said looped wall having an inner wall surface defining said finger passage, and an outer wall surface opposite to said inner wall surface, each of said looped flanges projecting from a respective one of said inner and outer peripheries of said looped wall away from said inner wall surface.

8. The auxiliary device of claim 7, wherein said looped flanges are adapted to fix to the inner and outer surfaces of the protective sleeve, respectively.

9. The auxiliary device of claim 7, wherein one of said looped flanges has a looped stitch groove formed in an outer surface thereof.
